(19) United States
(12) Patent Application Publication
(10) Pub. No.: US 2023/0021368 A1
(43) Pub. Date: Jan. 26, 2023

(54) PROCESS FOR THE PREPARATION OF CHLORANTRANILIPROLE

(71) Applicant: Gharda Chemicals Limited, Maharashtra (IN)

(72) Inventors: Suchet Saran MATHUR, Maharashtra (IN); Hridaynath Vishwanath MHATRE, Maharashtra (IN); Vishal Parshuram PEDHAVI, Maharashtra (IN); Dinesh JAWALE, Maharashtra (IN)

(21) Appl. No.: 17/782,807

(22) PCT Filed: Nov. 19, 2020
(86) PCT No.: PCT/IB2020/060891

§ 371 Date: Jun. 6, 2022

(30) Foreign Application Priority Data
Nov. 19, 2019 (IN) 201921047193

(51) Int. Cl. C07D 401/04

(52) U.S. Cl. CPC C07D 401/04

(57) ABSTRACT

The present disclosure relates to a process for the preparation of Chlorantraniliprole. The process of the present disclosure is carried out at an ambient temperature by using an inorganic base which can be readily separated from Chlorantraniliprole. The process is simple, efficient, environment friendly, and provides Chlorantraniliprole with high purity and high yield.

### FIELD

The present disclosure relates to a process for the preparation of Chlorantraniliprole.

### BACKGROUND

The background information herein below relates to the present disclosure but is not necessarily prior art.

Chlorantraniliprole is a useful agrochemical. The structural formula for Chlorantraniliprole is as given below.

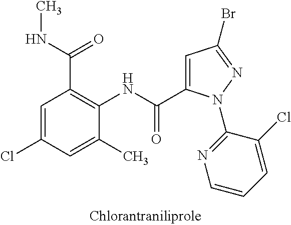 [SMILES: CNC(=O)c1cc(Cl)cc(C)c1NC(=O)c1cc(Br)nn1-c1ncccc1Cl]

Conventionally, the preparation of Chlorantraniliprole is carried out by using organic bases which are expensive and difficult to separate from the products.

There is, therefore, felt a need for a process for preparing Chlorantraniliprole that mitigates the drawbacks mentioned hereinabove.

### OBJECTS

Some of the objects of the present disclosure, which at least one embodiment herein satisfies, are as follows:

An object of the present disclosure is to ameliorate one or more problems of the prior art or to at least provide a useful alternative.

Another object of the present disclosure is to provide a process for the preparation of Chlorantraniliprole with greater yield and high purity.

Yet another object of the present disclosure is to provide a process for the preparation of Chlorantraniliprole that is simple, efficient, and economical.

Still another object of the present disclosure is to provide a process for the preparation of Chlorantraniliprole that is environment friendly.

Other objects and advantages of the present disclosure will be more apparent from the following description, which is not intended to limit the scope of the present disclosure.

### SUMMARY

The present disclosure relates to a process for preparing Chlorantraniliprole. The process comprises adding 3-bromo-1-(3-chloro-2-pyridinyl)-1H-pyrazole-5-carboxylic acid to a fluid medium to obtain a reaction mass. Further, reacting 3-bromo-1-(3-chloro-2-pyridinyl)-1H-pyrazole-5-carboxylic acid from the reaction mass with an inorganic base under stirring to obtain a slurry. The so obtained slurry is reacted with 2-amino-5-chloro-N,3-dimethylbenzamide under stirring, followed by adding sulfonyl chloride represented by R-SO₂Cl to obtain a reaction mixture. The reaction mixture is equilibrated for a predetermined time period to obtain Chlorantraniliprole. The complete process is carried out at a temperature in the range of 20° C. to 35° C.

### DETAILED DESCRIPTION

Embodiments are provided so as to thoroughly and fully convey the scope of the present disclosure to the person skilled in the art. Numerous details are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, known processes or well-known apparatus or structures, and well known techniques are not described in detail.

The terminology used, in the present disclosure, is only for the purpose of explaining a particular embodiment and such terminology shall not be considered to limit the scope of the present disclosure. As used in the present disclosure, the forms “a,” “an,” and “the” may be intended to include the plural forms as well, unless the context clearly suggests otherwise. The terms “comprises,” “comprising,” “including,” and “having,” are open ended transitional phrases and therefore specify the presence of stated features, integers, steps, operations, elements, modules, units and/or components, but do not forbid the presence or addition of one or more other features, integers, steps, operations, elements, components, and/or groups thereof. The particular order of steps disclosed in the method and process of the present disclosure are not to be construed as necessarily requiring their performance as described or illustrated. It is also to be understood that additional or alternative steps may be employed.

The terms first, second, third, etc., should not be construed to limit the scope of the present disclosure as the aforementioned terms may be only used to distinguish one element, component, region, layer or section from another component, region, layer or section. Terms such as first, second, third etc., when used herein do not imply a specific sequence or order unless clearly suggested by the present disclosure.

Chlorantraniliprole is a useful agrochemical. The structural formula for Chlorantraniliprole is as given below.

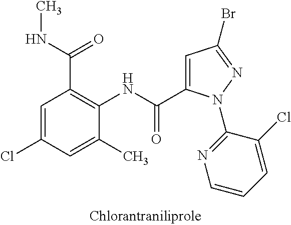 [SMILES: CNC(=O)c1cc(Cl)cc(C)c1NC(=O)c1cc(Br)nn1-c1ncccc1Cl]

The process of the present disclosure provides a simple, environment friendly, and economical process that, results in improved yields and higher purity of the final product.

The process is described in detail.

In the first step, a predetermined amount of 3-bromo-1-(3-chloro-2-pyridinyl)-1H-pyrazole-5-carboxylic acid is added to a fluid medium to obtain a reaction mass.

In accordance with one embodiment, the fluid medium is selected from the group consisting of acetonitrile, methylene dichloride, methyl ethyl ketone, methanol, ethanol, n-propanol, isopropanol, n-butanol, iso-butanol, and tert-butanol.

In an exemplary embodiment of the present disclosure, the fluid medium is acetonitrile. In another exemplary embodiment of the present disclosure, the fluid medium is methyl ethyl ketone. In yet another exemplary embodiment of the present disclosure, the fluid medium is isopropanol. In still another exemplary embodiment of the present disclosure, the fluid medium is methylene dichloride.

The predetermined amount of 3-bromo-1-(3-chloro-2-pyridinyl)-1H-pyrazole-5-carboxylic acid is in the range of 1:5 to 1:10 (w/v) with respect to the total volume of the fluid medium.

In the second step, a predetermined amount of an inorganic base is added to the reaction mass under stirring to obtain a slurry.

In an embodiment, the inorganic base is selected from potassium carbonate, sodium carbonate, sodium bicarbonate, potassium bicarbonate, sodium hydroxide, potassium hydroxide, magnesium carbonate, magnesium hydroxide, lithium carbonate, lithium hydroxide monohydrate, caesium carbonate calcium carbonate, and calcium hydroxide.

The predetermined amount of the base used in the process of the present disclosure is in the range of 1:1 to 1:4 (m/m) with respect to the amount of 3-bromo-1-(3-chloro-2-pyridinyl)-1H-pyrazole-5-carboxylic acid. In an exemplary embodiment, the predetermined amount is 1:1.3 (m/m).

In the third step, the slurry is reacted with 2-amino-5-chloro-N,3-dimethylbenzamide under stirring, followed by adding sulfonyl chloride represented by R—SO₂Cl to obtain a reaction mixture, wherein R is C1-C4 alkyl, C1-C2 haloalkyl, phenyl optionally substituted with 1-3 substituent selected from the group consisting of halogen, C1-C3 alkyl, and nitro. In an exemplary embodiment, the sulfonyl chloride is methanesulfonyl chloride. In another exemplary embodiment, the sulfonyl chloride is p-methyl-benzenesulfonyl chloride. In yet another exemplary embodiment, the sulfonyl chloride is p-chlorobenzenesulfonyl chloride. In still another exemplary embodiment, the sulfonyl chloride is m-nitrobenzenesulfonyl chloride.

In an embodiment, the amount of 2-amino-5-chloro-N,3-dimethylbenzamide is in the range of 1:1 to 1:1.2 (m/m) with respect of the amount of the sulfonyl chloride. In an exemplary embodiment, the amount of 2-amino-5-chloro-N,3-dimethylbenzamide is 1:1.18 (m/m) with respect of the methane sulfonyl chloride. In another exemplary embodiment, the amount of 2-amino-5-chloro-N,3-dimethylbenzamide is 1:1.11 (m/m) with respect of the p-methyl-benzenesulfonyl chloride.

In the final step, the reaction mixture is equilibrated for a predetermined time period to obtain Chlorantraniliprole.

In an embodiment, the process i.e. from the first step to the final step is carried out at a temperature in the range of 20° C. to 35° C. The process of the present disclosure is carried out at an ambient temperature, hence the process is economical.

In one embodiment, the predetermined time period for equilibration is in the range of 1 hour to 5 hours. In an exemplary embodiment, the predetermined time period for equilibration is 1 hour. In another exemplary embodiment, the predetermined time period for equilibration is 4 hours.

In an exemplary embodiment, the process of the present disclosure for preparing Chlorantraniliprole involves the following steps:
a) adding 3-bromo-1-(3-chloro-2-pyridinyl)-1H-pyrazole-5-carboxylic acid to a fluid medium to obtain a reaction mass;
b) reacting 3-bromo-1-(3-chloro-2-pyridinyl)-1H-pyrazole-5-carboxylic acid from the mixture with an inorganic base under stirring to obtain a slurry;
c) reacting the slurry with 2-amino-5-chloro-N,3-dimethylbenzamide under stirring, followed by adding sulfonyl chloride represented by R—SO₂Cl to obtain a reaction mixture; and
d) equilibrating the reaction mixture for a predetermined time period to obtain Chlorantraniliprole,
   wherein the process steps a) to d) is carried out at a temperature in the range of 20° C. to 35° C.

The schematic representation of the process for the preparation of Chlorantraniliprole is as given below:

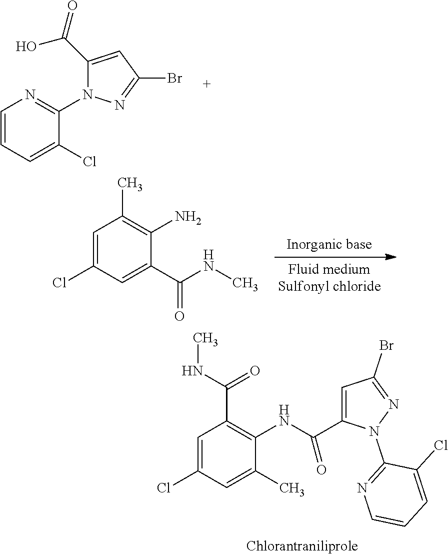

In an embodiment, the reaction mixture is monitored by HPLC for the formation of Chlorantraniliprole. The so obtained Chlorantraniliprole is filtered and washed with acetonitrile to obtain a cake. The cake is added into water under stirring to obtain a slurry. The slurry is filtered, washed with water, and dried to obtain Chlorantraniliprole.

The present disclosure provides an alternative method for preparing Chlorantraniliprole by using an inorganic base which increases the yield and purity of Chlorantraniliprole. The inorganic base is easily available and inexpensive. The inorganic base used in the process of the present disclosure can be easily separated by extracting from the reaction medium with an aqueous fluid medium. As a result of using the inorganic bases, the process of the present disclosure is cost-efficient and economical.

Thus, the present disclosure avoids the use of expensive organic bases and further avoids the use of expensive organic solvents for extraction of these organic bases from Chlorantraniliprole. Hence, the present disclosure saves on the cost of procuring expensive organic solvents and makes the process efficient, economic, and environment friendly.

Further, the sulfonyl chloride represented by R—SO₂Cl and the fluid medium can be separated, recovered, and recycled from the reaction medium. Hence, the process of the present disclosure is environment friendly.

The foregoing description of the embodiments has been provided for purposes of illustration and not intended to limit the scope of the present disclosure. Individual components of a particular embodiment are generally not limited to that particular embodiment, but, are interchangeable. Such variations are not to be regarded as a departure from the present disclosure, and all such modifications are considered to be within the scope of the present disclosure.

The present disclosure is further illustrated herein below with the help of the following experiments. The experiments used herein are intended merely to facilitate an understanding of the ways in which the embodiments herein may be practiced and to further enable those of skill in the art to practice the embodiments herein. Accordingly, the experiments should not be construed as limiting the scope of embodiments herein. These laboratory scale experiments can be scaled up to industrial/commercial scale and the results obtained can be extrapolated to industrial/commercial scale.

### EXPERIMENTAL DETAILS

### Example 1: Preparation of Chlorantraniliprole

3-bromo-1-(3-chloro-2-pyridinyl)-1H-pyrazole-5-carboxylic acid (32 gms) was added to acetonitrile (150 ml) under stirring to form a reaction mass. Potassium carbonate (16.6 gms; 0.12 mole) was added to the reaction mass under stirring to form a slurry. 2-amino-5-chloro-N,3-dimethylbenzamide (20 gms) was added to the slurry under stirring and rinsed with acetonitrile (25 ml). Methane sulfonyl chloride (14 gms; 0.12 moles) was added while maintaining the temperature at 25° C. to form a reaction mixture. This reaction mixture was equilibrated at 25° C. for 1 hour and was monitored by HPLC for the formation of Chlorantraniliprole. The so obtained Chlorantraniliprole was filtered at 32° C. and washed with acetonitrile to obtain a cake. The so obtained cake was made into slurry in water (70 ml) at 30° C. and filtered. It was further washed with water and dried to obtain Chlorantraniliprole of 95% purity and 83% yield.

### Examples 2 to 10: Preparation of Chlorantraniliprole

The Chlorantraniliprole was prepared similar to the process disclosed in Example 1 by varying the base and solvent, the results are provided below in Table 1.

TABLE 1

| Example | | | Purity | Yield on |
|---|---|---|---|---|
| No. | Base | Solvent | (%) | purity (%) |
| 2 | Na₂CO₃ | Acetonitrile | 95 | 67 |
| 3 | NaHCO₃ | Acetonitrile | 96 | 80 |
| 4 | KHCO₃ | Acetonitrile | 94 | 74 |
| 5 | NaOH | Acetonitrile | 96 | 52 |
| 6 | KOH | Acetonitrile | 95 | 39 |
| 7 | K₂CO₃ | Methylene dichloride | 87 | 58 |
| | | (MDC) | | |
| 8 | K₂CO₃ | Methyl Ethyl Ketone | 98 | 73 |
| | | (MEK) | | |
| 9 | K₂CO₃ | Aq-IPA | 96 | 40 |
| 10 | K₂CO₃ | Isopropanol (IPA) | 93 | 81 |

### Example 11: Preparation of Chlorantraniliprole

85 ml of acetonitrile was charged into the reactor followed by the addition of 3-Bromo-1-(3-chloro-2-pyridinyl)-1H-pyrazole-5-carboxylic acid (15.6 gms; purity 97.84%) under stirring to obtain a reaction mass. Potassium carbonate (8.4 gms; 0.06 mole) was added to the reaction mass under stirring to form a stirrable slurry. 2-Amino-5-chloro-N,3-dimethylbenzamide (10 gms; purity 99.2%) was added to this slurry under stirring and maintained at a temperature of 30° C. for 30 minutes to obtain a reaction mixture. Maintaining the temperature, p-methyl-benzenesulfonyl chloride (10.8 gms; purity 98%) solution in 15 ml of acetonitrile, was slowly added over a period of 30 minutes to the reaction mixture, to form a mixture. The mixture was equilibrated at 29° C. for 4 hours and was monitored by HPLC for the formation of Chlorantraniliprole. The so obtained Chlorantraniliprole was filtered at 28° C. and washed twice with 25 ml acetonitrile to obtain a cake. The so obtained cake was made into slurry in water (50 ml) at 28° C. and filtered. It was further washed with water and dried to obtain Chlorantraniliprole of 96.25% purity and 64% yield.

### Example 12: Preparation of Chlorantraniliprole

85 ml of acetonitrile was charged into the reactor followed by the addition of 3-Bromo-1-(3-chloro-2-pyridinyl)-1H-pyrazole-5-carboxylic acid (15.6 gms; purity 97.19%) under stirring to obtain a reaction mass. Potassium carbonate (8.28 gms; 0.06 mole) was added to the reaction mass under stirring to form a stirrable slurry. 2-Amino-5-chloro-N,3-dimethylbenzamide (10 gms; purity 98.12%) was added to this slurry under stirring and maintained at a temperature of 30° C. to 32° C. for 30 minutes to obtain a reaction mixture. Maintaining the temperature, p-chlorobenzenesulfonyl chloride (11.6 gms; purity 99%) solution in 15 ml of acetonitrile was slowly added over a period of 30 minutes to the reaction mixture slurry to form a mixture. The mixture was equilibrated at 29° C. for 4 hours and was monitored by HPLC for the formation of Chlorantraniliprole. The so obtained Chlorantraniliprole was filtered at 28° C. and washed twice with 20 ml acetonitrile to obtain a cake. The so obtained cake was made into a slurry in water (100 ml) at 28° C. and filtered. It was further washed twice with 25 ml water and dried to obtain Chlorantraniliprole of 97% purity and 86% yield.

### Example 13: Preparation of Chlorantraniliprole

80 ml of acetonitrile was charged into the reactor followed by the addition of 3-Bromo-1-(3-chloro-2-pyridinyl)-1H-pyrazole-5-carboxylic acid (15.6 gms; purity 97.84%) under stirring to obtain a reaction mass. Potassium carbonate (8.4 gms; 0.06 mole) was added to the reaction mass under stirring to form a stirrable slurry. 2-Amino-5-chloro-N,3-dimethylbenzamide (10 gms; purity 98.12%) was added to this slurry under stirring and maintained at a temperature of 30° C. to 32° C. for 30 minutes to obtain a reaction mixture. Maintaining the temperature, m-nitrobenzenesulfonyl chloride (12.4 gms; purity 99%) solution in 20 ml of acetonitrile was slowly added over a period of 45 minutes to the reaction mixture to form a thick mixture. To the obtained thick mixture, 25 ml of acetonitrile was added to form a stirrable reaction mixture. The stirrable reaction mixture was equilibrated at 29° C. for 4 hours and was monitored by HPLC for the formation of Chlorantraniliprole (73%). The so obtained Chlorantraniliprole was stirred with 500 ml water at room temperature for 30 min, followed by filtration, and washing twice with 25 ml water, and dried to obtain Chlorantraniliprole of 98.3% purity and 77% yield.

### Technical Advancement and Economic Significance

The present disclosure described herein above has several technical advantages including, but not limited to, the realization of a process for the preparation of Chlorantraniliprole, wherein:
the inorganic base used is inexpensive and can be separated;
the fluid medium and sulfonyl chloride used in the process can be separated, recovered, and recycled;
the reaction is carried out at ambient temperature;
the process provides Chlorantraniliprole with high purity and in high yield; and
the process is simple, efficient, and environment friendly.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

The use of the expression “at least” or “at least one” suggests the use of one or more elements or ingredients or quantities, as the use may be in the embodiment of the disclosure to achieve one or more of the desired objects or results.

Any discussion of documents, acts, materials, devices, articles or the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values mentioned for the various physical parameters, dimensions or quantities are only approximations and it is envisaged that the values higher/lower than the numerical values assigned to the parameters, dimensions or quantities fall within the scope of the disclosure, unless there is a statement in the specification specific to the contrary.

While considerable emphasis has been placed herein on the components and component parts of the preferred embodiments, it will be appreciated that many embodiments can be made and that many changes can be made in the preferred embodiments without departing from the principles of the disclosure. These and other changes in the preferred embodiment as well as other embodiments of the disclosure will be apparent to those skilled in the art from the disclosure herein, whereby it is to be distinctly understood that the foregoing descriptive matter is to be interpreted merely as illustrative of the disclosure and not as a limitation.

1. A process for the preparation of Chlorantraniliprole represented by formula (I): 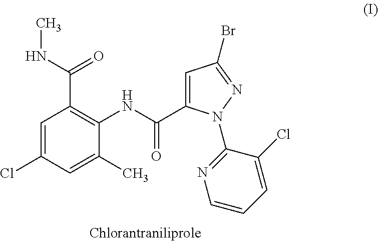 [SMILES: CNC(=O)c1cc(Cl)cc(C)c1NC(=O)c1cc(Br)nn1-c1ncccc1Cl]
said process comprising the following steps:
a) adding 3-bromo-1-(3-chloro-2-pyridinyl)-1H-pyrazole-5-carboxylic acid to a fluid medium to obtain a reaction mass;
b) reacting 3-bromo-1-(3-chloro-2-pyridinyl)-1H-pyrazole-5-carboxylic acid from said reaction mass with an inorganic base under stirring to obtain a slurry;
c) reacting said slurry with 2-amino-5-chloro-N,3-dimethylbenzamide under stirring, followed by adding sulfonyl chloride represented by R—SO₂Cl to obtain a reaction mixture,
wherein R is C1-C4 alkyl, C1-C2 haloalkyl, phenyl optionally substituted with 1-3 substituent selected from the group consisting of halogen, C1-C3 alkyl, and nitro; and
d) equilibrating the reaction mixture for a predetermined time period to obtain Chlorantraniliprole of formula I,
wherein said process steps a) to d) is carried out at a temperature in the range of 20° C. to 35° C.

2. The process as claimed in claim 1, wherein said fluid medium is at least one selected from acetonitrile, methylene dichloride, methyl ethyl ketone, methanol, ethanol, n-propanol, isopropanol, n-butanol, iso-butanol, and tert-butanol.

3. The process as claimed in claim 1, wherein said inorganic base is selected from potassium carbonate, sodium carbonate, sodium bicarbonate, potassium bicarbonate, sodium hydroxide, potassium hydroxide, magnesium carbonate, magnesium hydroxide, lithium carbonate, lithium hydroxide monohydrate, caesium carbonate, calcium carbonate, and calcium hydroxide.

4. The process as claimed in claim 1, wherein said inorganic base is potassium carbonate.

5. The process as claimed in claim 1, wherein said sulfonyl chloride is selected from methane sulfonyl chloride, p-methylbenzenesulfonyl chloride, p-chlorobenzenesulfonyl chloride, and m-nitrobenzenesulfonyl chloride.

6. The process as claimed in claim 1, wherein said predetermined time period in step d) is in the range of 1 to 5 hours.

7. The process as claimed in claim 1, wherein an amount of said base is in the range of 1:1 to 1:4 (m/m) with respect to the amount of 3-bromo-1-(3-chloro-2-pyridinyl)-1H-pyrazole-5-carboxylic acid.

8. The process as claimed in claim 1, wherein the amount of 2-amino-5-chloro-N,3-dimethylbenzamide is in the range of 1:1 to 1:1.2 (m/m) with respect to the amount of the sulfonyl chloride.
